(19) United States
(12) Patent Application Publication
(10) Pub. No.: US 2005/0260152 A1
(43) Pub. Date: Nov. 24, 2005

(54) Hair growth composition

(76) Inventor: Khalil Charlie Jaghab, Canton, MI (US)

Correspondence Address: YOUNG & BASILE, P.C., 3001 WEST BIG BEAVER ROAD, SUITE 624, TROY, MI, 48084, US

(21) Appl. No.: 11/120,881

(22) Filed: May 3, 2005

Related U.S. Application Data
(60) Provisional application No. 60/567,730, filed on May 3, 2004.

(51) Int. Cl. A61K 35/78; A61K 7/06

(52) U.S. Cl. 424/74; 424/754; 424/727

(57) ABSTRACT

A hair growth solution includes garlic dissolved in a liquid carrier which includes water, polysorbate, and additional components. The garlic, polysorbate, and water solution is mixed with another solution consisting of alcohol, camphor, salicylic acid, vitamin E, at least one of saw palmetto, glycenhetinic acid, pantothenyl alcohol, and a fragrance. The two component solutions are separately formed by mixing the individual constituents until a clear solution is formed. The two component solutions are then mixed together.

### CROSS-REFERENCE TO CO-PENDING APPLICATION

This application claims the benefit of the May 3, 2004, filing date of U.S. Provisional Application 60/567,730, the contents of which are incorporated herein in their entirety.

### BACKGROUND

The present invention relates, in general, to compositions, products and methods for promoting hair growth and preventing or minimizing hair loss.

Continuing efforts have been made over a long period of time to develop compositions or products which prevent or minimize hair loss and which stimulate hair growth or regrowth. These efforts have met with results ranging from complete failure to varying degrees of success.

Over the counter, topically administered drugs approved by the United States Food and Drug Administration are currently available. These products exhibit some degree of success in promoting and/or restoring hair growth, particularly on the crown of a human scalp. However, these products do not restore hair growth on all individuals and the degree of hair regrowth on individuals which show some degree of hair growth varies considerably.

Thus, there is still a need for a composition for promoting hair growth and/or preventing or minimizing hair loss that provides a higher degree of hair growth on a wider range of individuals.

### SUMMARY

A hair growth composition includes garlic as an active component in a liquid carrier as a topical application.

In one aspect, the solution includes about 5.6% by weight of garlic and about 1.0% polysorbate dissolved in water.

In another aspect, the hair growth solution also includes about 35.7% ethanol, about 0.1% camphor, about 0.10% salicylic acid, about 0.01% Vitamin E oil, about 2.0% saw palmetto root extract, and about 1.7% fragrance.

The hair growth solution may also include, by weight percentage, about 0.10% salicylic acid, about 0.10% Vitamin E oil, about 2.0% saw palmetto root extract, and about 1.7% fragrance.

In another aspect, the solution further comprises, by weight, about 0.2% salicylic acid, about 0.1% camphor, about 0.2% pantothenyl alcohol, about 0.01% 18 beta-Glycerrhetinic acid, about 0.01% alpha-Tocopherol acetate, and about 0.2% Cool Water fragrance.

In another aspect, the hair growth solution includes, by weight, about 0.1% camphor, about 0.2% salicylic acid and about 0.01% Vitamin E oil.

The present hair growth solution is formed of a mixture of two component solutions. The first component part or solution is formed on camphor, salicylic acid, vitamin E, FDA ethanol, and at least one of saw palmetto root extract, glycenhetinic acid, and pantothenyl alcohol. The second component part or solution includes water in quantity sufficient, garlic, polysorbate, and at least one of licorice testosterone acid, fragrance, glycenhetinic acid, capsaicin, and ursolic acid.

The hair growth composition of the present invention provides effective hair growth which can be easily topically applied.

### DETAILED DESCRIPTION

The present composition for stimulating hair growth and/or preventing or minimizing hair loss includes a topical application of a composition including garlic as an active component.

More particularly, the present hair growth or recovery composition utilizes a mixture of a garlic component and a polysorbate component, such as polysorbate 80.

The present composition utilizes a garlic and polysorbate component which is dissolved and mixed in a carrier composition which is designed to minimize precipitation and to provide an esthetically pleasing fragrance and ease of application.

In one aspect of the invention, a first composition part formed of ethanol, camphor, salicylic acid, Vitamin E, saw palmetto root extract and a fragrance is mixed until a clear solution is formed. A second composition part formed of tap water and a mixture of garlic, either garlic juice, garlic oil or garlic extract (concentrate), and polysorbate 80 is separately mixed. The first and second parts are then thoroughly mixed. The complete composition is allowed to sit for a period of time, to settle out any particulates. The composition may alternately be filtered to remove any precipitates.

### EXAMPLE 1

A first composition component is formed by mixing about 35.6% by weight of ethanol is mixed with about 0.10% camphor, about 0.10% salicylic acid, about 0.10% Vitamin E oil, about 2.0% saw palmetto root extract, and about 1.7% of appropriate fragrance, by example only. The ingredients are mixed until a clear solution is formed.

In a separate mixture, second composition component is formed by mixing about 54.5% d.i. water and about 6.6% of a mixture of garlic juice (garlic oil or garlic extract) and polysorbate until all components are solubulized in the water. The garlic component is about 5.6% and the polysorbate component is about 1.0%, all by weight.

Next, the first and second components are combined and mixed. The pH is adjusted, if necessary, to about 5.4-5.6 by adding a sufficient quantity of citric acid.

### EXAMPLE 2

A first composition component part is formed of a mixture of about 64% denatured alcohol (SDA 40B), about 0.2% salicylic acid, about 0.1% camphor, about 0.2% pantothenyl alcohol, 0.01% beta-Glycerrhetinic acid, about 0.01% DL-alpha-Tocopherol (Vitamin E), and about 0.2% appropriate fragrance are mixed until all parts are solubulized in the alcohol carrier.

A second composition component part is formed of about 34.8% d.i. water and about 0.2% garlic iodide extract. The garlic iodide extract is mixed in the d.i. water until totally dissolved.

The second component part is then slowly mixed into the first component part to form a clear solution. The pH is adjusted, if necessary, to about 5.4-5.6 by adding a sufficient quantity of citric acid.

### EXAMPLE 3

A first component part includes about 0.1% camphor, about 0.2% salicylic acid and 0.01% Vitamin E oil which are mixed in about 30% by weight of FDA ethanol. The camphor and salicylic acid are slowly added to the ethanol and mixed until solubulized. The Vitamin E oil is then added and mixed until solubulized.

A second component part includes about 2.0% testosterone extract which is mixed with about 2.0% licorice extract about 5.0% polysorbate, and 2.5% garlic oil and about 1.5% Cool Water scent (International Flavors and Fragrance) in a quantity sufficient of d.i. water, i.e., about 56.5% by weight, until all of the components are completely dissolved. Specifically, the polysorbate is added to the tap water and mixed to completely dissolve the polysorbate. The garlic oil is then added and solubulized. All of the other ingredients are then slowly added until solubulized.

The first and second component parts are then slowly mixed and vigorously stirred. The pH is adjusted, if necessary, to about 5.4-5.6 by adding a sufficient quantity of citric acid. If a fine precipitate forms during a settling period, the solution can be filtered to remove any fine particulates.

### EXAMPLE 4

A first component part includes about 30 to about 40% by weight of denatured alcohol (SDA) which is mixed with about 0.05-0.4% camphor, about 0.05-0.4% salicylic acid, about 0.05-0.4% DL-alpha-Tocopheryl (vitamin E), and about 0.05-0.4% saw palmetto root extract.

All of the individual parts of the first component are thoroughly mixed until a clear solution is formed. A second component part is separately formed by mixing about 1.5-7.0% garlic extract, about 0.05-0.7% capsaicin, about 0.05-5.0% ursolic acid, and minute quantities of glycyrrhetinic acid and polysorbate are mixed with the quantities sufficient of water until a clear solution is formed.

The first component part and the second component part are mixed. Fragrance of a quantity sufficient is then added. The pH is adjusted, if necessary, to about 5.4-5.6 by adding a sufficient quantity of citric acid.

Application:

The above-described hair growth composition is applied in small doses directly to a human scalp, either over the entire scalp or only in areas where hair regrowth is desired, such as the crown of the head. The application dose is applied evenly by means of a suitable applicator or by hand. The applied solution is then rubbed into the scalp.

In summary, the present invention is a hair growth solution comprising garlic dissolved in a liquid carrier formulated for direct application to a human scalp.

1. A hair growth solution comprising:
garlic solubulized in a liquid carrier.

2. The hair growth solution of claim 1 wherein the solution comprises:
by weight, about 5.6% garlic, and about 1.0% polysorbate dissolved in water.

3. The hair growth solution of claim 2 wherein:
the water comprises about 54.5% of a complete solution mixture.

4. The hair growth solution of claim 3 further comprising:
by weight, about 35.6% ethanol, about 0.1% camphor, about 0.10% salicylic acid, 0.01% Vitamin E oil, about 2.0% saw palmetto root extract, and about 1.6% fragrance.

5. The hair growth solution of claim 1 wherein the solution further comprises:
about 0.10% camphor.

6. The hair growth solution of claim 1 wherein the solution comprises, by weight:
about 0.10% salicylic acid, about 0.10% Vitamin E oil, about 2.0% saw palmetto root extract, and about 1.6% fragrance.

7. The hair growth solution of claim 1 wherein the solution further comprises, by weight:
about 0.2% salicylic acid, about 0.1% camphor, about 0.2% pantothenyl alcohol, 0.01% beta-Glycerrhetinic acid, 0.01% DL-alpha-Tocopherol, and about 0.2% fragrance.

8. The hair growth solution of claim 1 wherein the solution further comprises, by weight:
about 0.1% camphor, about 0.2% salicylic acid and about 0.01% Vitamin E oil.

9. The hair growth solution of claim 1 further comprising:
about 34.8% water and about 0.2% garlic.

10. The hair growth solution of claim 9 further comprising:
about 64.5% alcohol, about 0.1% camphor, about 0.2% salicylic acid, 0.01% Vitamin E, 0.01% glycenhetnic acid, and about 0.2% pantothenyl alcohol.

11. The hair growth solution of claim 1 further comprising:
about 56.5% water, about 2.5% garlic, about 5.0% polysorbate, about 2.0% licorice extract, about 2.0% testosterone extract, and about 1.5% fragrance.

12. The hair growth solution of claim 10 further comprising:
about 30.8% ethanol, about 0.1% camphor, about 0.2% salicylic acid, and about 0.1% Vitamin E.

13. The hair growth solution of claim 1 wherein the garlic content comprises about 1.5% to about 7.0% by weight garlic; and wherein the hair growth solution further comprises:
about 0.0 to about 7.0% capsaicin and about 0.5-5.0% ursolic acid with minute amounts of polysorbate and gylcenhetnic acid, water provided in the quantity sufficient.

14. The hair growth solution of claim 13 further comprising:
about 30-40% by weight alcohol, about 0.05-about 0.4% camphor, 0.05-0.4% salicylic acid, 0.05-0.4% Vitamin E, and 0.05-0.4% saw palmetto root extract.

15. The hair growth solution comprising:
first and second mixed component parts;
the first component part formed of one of alcohol and ethanol, camphor, salicylic acid, Vitamin E, and at least one of saw palmetto root extract, glycenhetinic acid and pantothenyl alcohol;
the second component part comprises water in quantity sufficient, garlic, polysorbate and at least one of licorice, testosterone acid, fragrance, glycenhetinic acid, capsaicin, and ursolic acid.
